(19) United States
(12) Patent Application Publication
(10) Pub. No.: US 2020/0375511 A1
(43) Pub. Date: Dec. 3, 2020

(54) TAILORED DRUG DELIVERY VEHICLES FOR IN VIVO PROTECTION OF ANALYTE SENSING COMPOUNDS

(71) Applicant: Senseonics, Incorporated, Germantown, MD (US)

(72) Inventors: Philip Huffstetler, Germantown, MD (US); Wendolyn Sandoval, Germantown, MD (US); Carrie R. Lorenz, Reston, VA (US); Venkata Velvadapu, Germantown, MD (US); Joon Chatterjee, Germantown, MD (US)

(73) Assignee: Senseonics, Incorporated, Germantown, MD (US)

(21) Appl. No.: 16/887,704

(22) Filed: May 29, 2020

Related U.S. Application Data
(60) Provisional application No. 62/854,064, filed on May 29, 2019.

(51) Int. Cl. A61B 5/145; A61B 5/00; A61K 31/573; A61K 47/12; A61K 47/60

(52) U.S. Cl. CPC A61B 5/14532; A61B 5/14503; A61B 5/6867; A61B 2562/16; A61K 47/12; A61K 47/60; A61B 2503/40; A61K 31/573

(57) ABSTRACT

A sensor (e.g., an optical sensor) that may be implanted within a living animal (e.g., a human) and may be used to measure an analyte (e.g., glucose or oxygen) in a medium (e.g., interstitial fluid, blood, or intraperitoneal fluid) within the animal. The sensor may include a sensor housing, an analyte indicator covering at least a portion of the sensor housing, and a drug eluting material having tailored elution properties that contains a drug that reduces deterioration of the analyte indicator, wherein the drug eluting material is incorporated in and/or in close proximity to the analyte indicator, and the drug eluting material is configured to release the drug according to a tailored elution profile.

### CROSS-REFERENCE TO RELATED APPLICATION

The present application claims the benefit of priority to U.S. Provisional Application Ser. No. 62/854,064, filed on May 29, 2019, which is incorporated herein by reference in its entirety.

### BACKGROUND

### Field of Invention

The present invention relates generally to continuous reduction of in vivo degradation of analyte sensor moieties when measuring an analyte in a medium of a living animal using a system including a sensor implanted (partially or fully) or inserted into the living animal. Specifically, the present invention relates to a sensor that utilizes a drug eluting matrix having a tailored drug elution profile to reduce degradation.

### Discussion of the Background

A sensor may be implanted (partially or fully) within a living animal (e.g., a human) and used to measure an analyte (e.g., glucose, oxygen, cardiac markers, low-density lipoprotein (LDL), high-density lipoprotein (HDL), or triglycerides) in a medium (e.g., interstitial fluid (ISF), blood, or intraperitoneal fluid) within the living animal. The sensor may include a light source (e.g., a light-emitting diode (LED) or other light emitting element), indicator molecules, and a photodetector (e.g., a photodiode, phototransistor, photoresistor or other photosensitive element). Examples of implantable sensors employing indicator molecules to measure an analyte are described in U.S. Pat. Nos. 5,517,313 and 5,512,246, which are incorporated herein by reference in their entirety.

A sensor may include an analyte indicator, which may be in the form of indicator molecules embedded in a graft (i.e., layer or matrix). For example, in an implantable fluorescence-based glucose sensor, fluorescent indicator molecules may reversibly bind glucose and, when irradiated with excitation light (e.g., light having a wavelength of approximately 378 nm), emit an amount of light (e.g., light in the range of 400 to 500 nm) that depends on whether glucose is bound to the indicator molecule.

If a sensor is implanted in the body of a living animal, the animal's immune system may begin to attack the sensor. For instance, if a sensor is implanted in a human, white blood cells may attack the sensor as a foreign body, and, in the initial immune system onslaught, neutrophils may be the primary white blood cells attacking the sensor. The defense mechanism of neutrophils includes the release of highly caustic substances known as reactive oxygen species. The reactive oxygen species include, for example, hydrogen peroxide. As used herein, the terms “degradative species” and “biological oxidizers” generally refer to reactive physiological molecules and radicals that degrade the indicator molecules.

Hydrogen peroxide and other degradative species such as reactive oxygen and nitrogen species may degrade the indicator molecules of an analyte indicator. For instance, in indicator molecules having a boronate group, hydrogen peroxide may degrade the indicator molecules by oxidizing the boronate group, thus disabling the ability of the indicator molecule to bind glucose. The longevity of certain implantable sensors is achieved in part or in whole using anti-inflammatory drugs such as dexamethasone. In conventional sensors that use anti-inflammatory drugs, there is a constant rate of drug elution for patients with both low- and elevated-levels of oxidative stress. As such, the drug is not effectively utilized, leading to a short than desired sensor lifetime.

There is presently a need in the art for improvements in reducing analyte indicator degradation. There is also a need in the art for continuous analyte sensors having increased longevity.

### SUMMARY

The present invention overcomes the disadvantages of prior systems by providing, among other advantages, reduced analyte indicator degradation.

In one aspect, the present disclosure provides a sensor for measurement of an analyte in a medium within a living animal. In one aspect, a sensor according to the present disclosure may include a sensor housing, an analyte indicator covering at least a portion of the sensor housing, and a drug eluting material having tailored elution properties. In some aspects, a drug eluting material according to the present disclosure may include a drug that reduces deterioration of the analyte indicator, wherein the drug eluting material is incorporated in and/or in close proximity to the analyte indicator, and the drug eluting material is configured to release the drug according to a tailored elution profile.

In one aspect, the present disclosure provides a method of fabricating a sensor for measurement of an analyte in a medium within a living animal by applying an analyte indicator to a sensor housing of the sensor such that the applied analyte indicator covers at least a portion of the sensor housing, and applying a drug eluting material having tailored elution properties comprising a drug that reduces deterioration of the analyte indicator such that the applied drug eluting material is incorporated to the sensor in and/or in close proximity to the analyte indicator, wherein the drug eluting material is configured to release the drug according to a tailored elution profile and reduce deterioration of the analyte indicator.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting examples of the present invention. In the drawings, like reference numbers indicate identical or functionally similar elements.

FIG. 1 is a schematic view illustrating a sensor system embodying aspects of the present invention.

FIG. 2 illustrates a perspective view of a sensor embodying aspects of the present invention.

FIG. 3 illustrates an exploded view of a sensor embodying aspects of the present invention.

FIG. 4 illustrates a sensor having a dip coated drug-eluting polymer matrix embodying aspects of the present invention.

FIGS. 5A-5E illustrate examples of sensors having a preformed drug-eluting polymer matrix embodying aspects of the present invention.

FIG. 6 illustrates two exemplary and non-limiting steady state release profiles of the present invention.

FIG. 7 illustrates two exemplary and non-limiting tailored elution profiles of the present invention.

### DETAILED DESCRIPTION

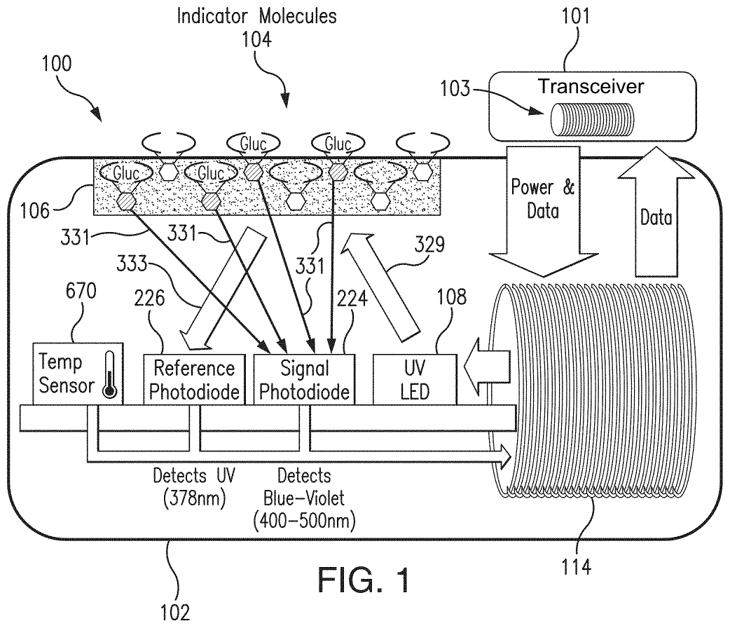

FIG. 1 is a schematic view of a sensor system embodying aspects of the present invention. In some non-limiting example, as shown in FIG. 1, the system may include a sensor 100 and an external transceiver 101. In some examples, the sensor 100 may be an implantable sensor configured to be fully or partially implanted in a living animal (e.g., a living human). The sensor 100 may be implanted, for example, in a living animal's arm, wrist, leg, abdomen, peritoneum, or other region of the living animal suitable for sensor implantation. For example, in some non-limiting examples, the sensor 100 may be implanted beneath the skin (i.e., in the subcutaneous or peritoneal tissues). However, this is not required, and, in some alternative examples, the sensor 100 may be a transcutaneous sensor.

In some aspects, a transceiver 101 may be an electronic device that communicates with the sensor 100 to power the sensor 100, provide commands and/or data to the sensor 100, and/or receive data from the sensor 100. In some aspects, the received data may include one or more sensor measurements. In some aspects, the sensor measurements may include, for example and without limitation, one or more light measurements from one or more photodetectors of the sensor 100 and/or one or more temperature measurements from one or more temperature sensors of the sensor 100. In some aspects, the transceiver 101 may calculate analyte (e.g., glucose) concentrations from the measurement information received from the sensor 100.

In some non-limiting aspects, the transceiver 101 may be a handheld device or an on-body/wearable device. For example, in some aspects where the transceiver 101 is an on-body/wearable device, the transceiver 101 may be held in place by a band (e.g., an armband or wristband) and/or adhesive, and the transceiver 101 may convey (e.g., periodically, such as every two minutes, and/or upon user initiation) measurement commands (i.e., requests for measurement information) to the sensor 100. In some aspects where the transceiver 101 is a handheld device, positioning (i.e., hovering or swiping/waving/passing) the transceiver 101 within range over the sensor implant site (i.e., within proximity of the sensor 100) may cause the transceiver 101 to automatically convey a measurement command to the sensor 100 and receive a data from the sensor 100.

In some aspects, as shown in FIG. 1, the transceiver 101 may include an inductive element 103, such as, for example, a coil. In some aspects, the transceiver 101 may generate an electromagnetic wave or electrodynamic field (e.g., by using a coil) to induce a current in an inductive element 114 of the sensor 100. In some non-limiting aspects, the sensor 100 may use the current induced in the inductive element 114 to power the sensor 100. However, this is not required, and, in some alternative aspects, the sensor 100 may be powered by an internal power source (e.g., a battery).

In some aspects, the transceiver 101 may convey data (e.g., commands) to the sensor 100. For example, in some non-limiting aspects, the transceiver 101 may convey data by modulating the electromagnetic wave generated by the inductive element 103 (e.g., by modulating the current flowing through the inductive element 103 of the transceiver 101). In some aspects, the sensor 100 may detect/extract the modulation in the electromagnetic wave generated by the transceiver 101. Moreover, the transceiver 101 may receive data (e.g., one or more sensor measurements) from the sensor 100. For example, in some non-limiting aspects, the transceiver 101 may receive data by detecting modulations in the electromagnetic wave generated by the sensor 100, e.g., by detecting modulations in the current flowing through the inductive element 103 of the transceiver 101.

In some aspects, as shown in FIG. 1, the sensor 100 may include a sensor housing 102 (i.e., body, shell, capsule, or encasement), which may be rigid and biocompatible. In exemplary aspects, sensor housing 102 may be formed from a suitable, optically transmissive polymer material, such as, for example, acrylic polymers (e.g., polymethylmethacrylate (PMMA)).

In some aspects, as shown in FIG. 1, the sensor 100 may include an analyte indicator 106. In some non-limiting aspects, the analyte indicator 106 may be a polymer graft coated, diffused, adhered, or embedded on at least a portion of the exterior surface of the sensor housing 102. The analyte indicator 106 (e.g., polymer graft) may cover the entire surface of sensor housing 102 or only one or more portions of the surface of housing 102. As an alternative to coating the analyte indicator 106 on the outer surface of sensor housing 102, the analyte indicator 106 may be disposed on the outer surface of the sensor housing 102 in other ways, such as by deposition or adhesion. In some aspects, the analyte indicator 106 may be a fluorescent glucose indicating polymer. In one non-limiting aspect, the polymer is biocompatible and stable, grafted onto the surface of sensor housing 102, designed to allow for the direct measurement of glucose in interstitial fluid (ISF), blood, or intraperitoneal fluid after implantation of the sensor 100. In some aspects, the analyte indicator 106 may be a hydrogel.

In some aspects, the analyte indicator 106 (e.g., polymer graft) of the sensor 100 may include indicator molecules 104. The indicator molecules 104 may be distributed throughout the entire analyte indicator 106 or only throughout one or more portions of the analyte indicator 106. The indicator molecules 104 may be fluorescent indicator molecules (e.g., TFM having the chemical name 9-[N-[6-(4,4,5,5,-tetramethyl-1,3,2-dioxaborolano)-3-(trifluoromethyl)benzyl]-N-[3-(methacrylamido)propylamino]methyl]-10-[N-[6-(4,4,5,5,-tetramethyl-1,3,2-dioxaborolano)-3-(trifluoromethyl)benzyl]-N-[2-(carboxyethyl)amino]methyl]anthracene sodium salt) or light absorbing, non-fluorescent indicator molecules. In some aspects, the indicator molecules 104 may reversibly bind an analyte (e.g., glucose, oxygen, cardiac markers, low-density lipoprotein (LDL), high-density lipoprotein (HDL), or triglycerides). When an indicator molecule 104 has bound an analyte, the indicator molecule may become fluorescent, in which case the indicator molecule 104 is capable of absorbing (or being excited by) excitation light 329 and emitting light 331. In one non-limiting aspect, the excitation light 329 may have a wavelength of approximately 378 nm, and the emission light 331 may have a wavelength in the range of 400 to 500 nm. When no analyte is bound, the indicator molecule 104 may be only weakly fluorescent.

In some aspects, the sensor 100 may include a light source 108, which may be, for example, a light emitting diode (LED) or other light source that emits radiation, including radiation over a range of wavelengths that interact with the indicator molecules 104. In other words, the light source 108 may emit the excitation light 329 that is absorbed by the indicator molecules in the matrix layer/polymer 104. As noted above, in one non-limiting aspect, the light source 108 may emit excitation light 329 at a wavelength of approximately 378 nm.

In some aspects, the sensor 100 may also include one or more photodetectors (e.g., photodiodes, phototransistors, photoresistors or other photosensitive elements). For example, in the aspect illustrated in FIG. 1, sensor 100 has a first photodetector 224 and a second photodetector 226. However, this is not required, and, in some alternative aspects, the sensor 100 may only include the first photodetector 224. In the case of a fluorescence-based sensor, the one or more photodetectors may be sensitive to fluorescent light emitted by the indicator molecules 104 such that a signal is generated by a photodetector (e.g., photodetector 224) in response thereto that is indicative of the level of fluorescence of the indicator molecules and, thus, the amount of analyte of interest (e.g., glucose).

Some part of the excitation light 329 emitted by the light source 108 may be reflected from the analyte indicator 106 back into the sensor 100 as reflection light 333, and some part of the absorbed excitation light may be emitted as emitted (fluoresced) light 331. In one non-limiting aspect, the emitted light 331 may have a different wavelength than the wavelength of the excitation light 329. The reflected light 333 and emitted (fluoresced) light 331 may be absorbed by the one or more photodetectors (e.g., first and second photodetectors 224 and 226) within the body of the sensor 100.

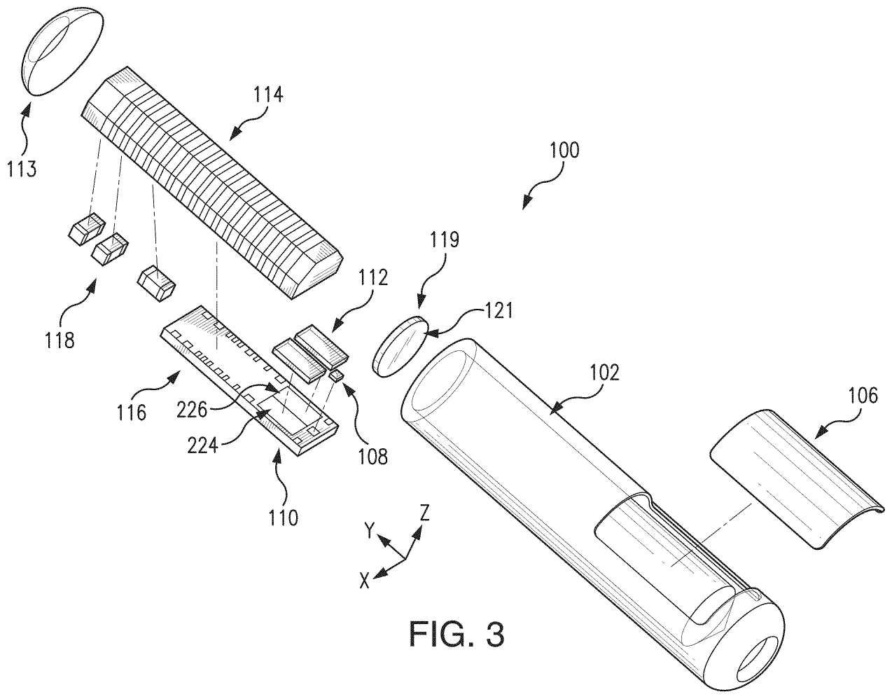

Each of the one or more photodetectors may be covered by a filter 112 (see FIG. 3) that allows only a certain subset of wavelengths of light to pass through. In some aspects, the one or more filters 112 may be thin glass filters. In some aspects, the one or more filters 112 may be thin film (e.g., dichroic) filters deposited on the glass and may pass only a narrow band of wavelengths and otherwise reflect most of the received light. In some aspects, the filters may be thin film (dichroic) filters deposited directly onto the photo detectors and may pass only a narrow band of wavelengths and otherwise reflect most of the light received thereby. The filters 112 may be identical (e.g., both filters 112 may allow signals to pass) or different (e.g., one filter 112 may be a reference filter and another filter 112 may be a signal filter).

In one non-limiting aspect, the second (reference) photodetector 226 may be covered by a reference photodiode filter that passes light at the same wavelength as is emitted from the light source 108 (e.g., 378 nm). The first (signal) photodetector 224 may detect the amount of fluoresced light 331 that is emitted from the molecules 104 in the analyte indicator 106. In one non-limiting aspect, the peak emission of the indicator molecules 104 may occur around 435 nm, and the first photodetector 224 may be covered by a signal filter that passes light in the range of about 400 nm to 500 nm. In some aspects, higher glucose levels/concentrations correspond to a greater amount of fluorescence of the molecules 104 in the analyte indicator 106, and, therefore, a greater number of photons striking the first photodetector 224.

In some aspects, as shown in FIG. 1, the sensor 100 may include a substrate 116. In some aspects, the substrate 116 may be a circuit board (e.g., a printed circuit board (PCB) or flexible PCB) on which circuit components (e.g., analog and/or digital circuit components) may be mounted or otherwise attached. However, in some alternative aspects, the substrate 116 may be a semiconductor substrate having circuitry fabricated therein. The circuitry may include analog and/or digital circuitry. Also, in some semiconductor substrate aspects, in addition to the circuitry fabricated in the semiconductor substrate, circuitry may be mounted or otherwise attached to the semiconductor substrate 116. In other words, in some semiconductor substrate aspects, a portion or all of the circuitry, which may include discrete circuit elements, an integrated circuit (e.g., an application specific integrated circuit (ASIC)) and/or other electronic components, may be fabricated in the semiconductor substrate 116 with the remainder of the circuitry is secured to the semiconductor substrate 116, which may provide communication paths between the various secured components.

In some aspects, the one or more of the sensor housing 102, analyte indicator 106, indicator molecules 104, light source 108, photodetectors 224, 226, temperature transducer 670, substrate 116, and inductive element 114 of sensor 100 may include some or all of the features described in one or more of U.S. application Ser. No. 13/761,839, filed on Feb. 7, 2013, U.S. application Ser. No. 13/937,871, filed on Jul. 9, 2013, and U.S. application Ser. No. 13/650,016, filed on Oct. 11, 2012, all of which are incorporated by reference in their entireties. Similarly, the structure and/or function of the sensor 100 and/or transceiver 101 may be as described in one or more of U.S. application Ser. Nos. 13/761,839, 13/937,871, and 13/650,016.

In some aspects, the sensor 100 may include a transceiver interface device, and the transceiver 101 may include a sensor interface device. In some aspects where the sensor 100 and transceiver 101 include an antenna or antennas (e.g., inductive elements 103 and 114), the transceiver interface device may include the inductive element 114 of the sensor 100, and the sensor interface device may include the inductive element 103 of the transceiver 101. In some of the transcutaneous aspects where there exists a wired connection between the sensor 100 and the transceiver 101, the transceiver interface device and sensor interface device may include the wired connection.

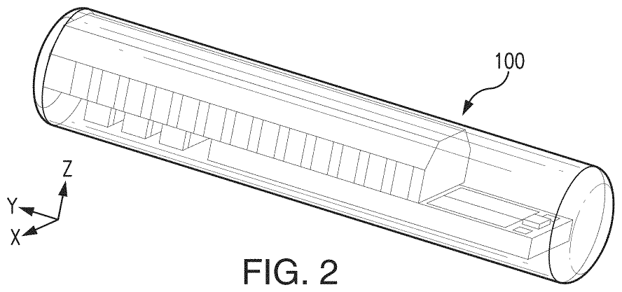

FIGS. 2 and 3 illustrate a non-limiting aspect of a sensor 100 embodying aspects of the present invention that may be used in the sensor system illustrated in FIG. 1. FIGS. 2 and 3 illustrate perspective and exploded views, respectively, of the non-limiting aspect of the sensor 100.

In some aspects, as illustrated in FIG. 3, the sensor housing 102 may include an end cap 113. In some aspects, the sensor 100 may include one or more capacitors 118. The one or more capacitors 118 may be, for example, one or more tuning capacitors and/or one or more regulation capacitors. The one or more capacitors 118 may be too large for fabrication in the semiconductor substrate 116 to be practical. Further, the one or more capacitors 118 may be in addition to one or more capacitors fabricated in the semiconductor substrate 116.

In some aspects, as illustrated in FIG. 3, the sensor 100 may include a reflector 119 (i.e., mirror). Reflector 119 may be attached to the semiconductor substrate 116 at an end thereof. In a non-limiting aspect, reflector 119 may be attached to the semiconductor substrate 116 so that a face portion 121 of reflector 119 is generally perpendicular to a top side of the semiconductor substrate 116 (i.e., the side of semiconductor substrate 116 on or in which the light source 108 and one or more photodetectors 110 are mounted or fabricated) and faces the light source 108. The face 121 of the reflector 119 may reflect radiation emitted by light source 108. In other words, the reflector 119 may block radiation emitted by light source 108 from exiting the axial end of the sensor 100.

According to one aspect of the invention, an application for which the sensor 100 was developed (although by no means the only application for which it is suitable) is measuring various biological analytes in the living body of an animal (including a human). For example, sensor 100 may be used to measure glucose, oxygen, toxins, pharmaceuticals or other drugs, hormones, and other metabolic analytes in, for example, the human body.

In some aspects, the specific composition of the analyte indicator 106 and the indicator molecules 104 may vary depending on the particular analyte the sensor is to be used to detect and/or where the sensor is to be used to detect the analyte (e.g., in the in subcutaneous tissues, blood, or peritoneum). In some aspects, the analyte indicator 106 facilitates exposure of the indicator molecules 104 to the analyte. In some aspects, the indicator molecules 104 may exhibit a characteristic (e.g., emit an amount of fluorescence light) that is a function of the concentration of the specific analyte to which the indicator molecules 104 are exposed.

The implantation or insertion of a medical device, such as a bio-sensor, into a user/patient's body can cause the body to exhibit adverse physiological reactions that are detrimental to the functioning of the device. The reactions may range from infections due to implantation surgery to the immunological response of a foreign object implanted in the body. That is, the performance of the implantable bio-sensor can be hindered or permanently damaged in vivo via the immunological response to an infection or the device itself. In particular, the performance of the analyte indicator 106 may be deteriorated by the immunological response of the body into which the sensor 100 is implanted. For example, as explained above, white blood cells, including neutrophils, may attack an implanted sensor 100. The neutrophils release degradative species including, inter alia, hydrogen peroxide, which may degrade indicator molecules 104 (e.g., by oxidizing a boronate group of an indicator molecule 104 and disabling the ability of the indicator molecule 104 to bind glucose and/or fluoresce). In some aspects, degradative species may include one or more of hydrogen peroxide, a reactive oxygen species, a reactive nitrogen species, and a free radical.

In some aspects, the sensor 100 may include one or more tailored drug-eluting materials comprising, e.g., matrices, membranes, hydrogels and/or polymers. In one aspect, the sensor 100 may include a drug eluting material covering at least a portion of the sensor housing 102. One or more therapeutic agents may be dispersed within a drug eluting material configured to provide tailored elution of the one or more therapeutic agents. In an alternative or additional aspect, one or more therapeutic agents may alternatively or additionally be incorporated within analyte indicator 106 and/or a membrane covering at least a portion of the analyte indicator as described in U.S. Pat. No. 9,931,068 (Huffstetler et al.), which is incorporated herein by reference in its entirety.

In one aspect, the drug eluting material, e.g., comprising a drug eluting polymer matrix, is configured to tailor the concentration of the drug as a function of distance from the indicator molecules 104 to protect the indicator molecules 104 from degradative species over time and prolong longevity of the sensor 100 when implanted in the body.

In one aspect, the drug eluting material, e.g., comprising a drug eluting polymer matrix, may include an additive providing tailored elution of the one or more therapeutic agents. In non-limiting examples of the disclosure, such additives may include a hydroxypropyl methylcellulose, a polyalkylene glycol, e.g., polyethylene glycol or polypropylene glycol, a polyalkylene oxide, e.g., polyethylene oxide or polypropylene oxide, polyethers and copolymers thereof, di-block, tri-block, grafted, post-functionalized polyether-siloxane copolymer systems, and combinations thereof. Non-limiting examples of block polymers that may be used include acryloxy block copolymers, e.g., acryloxy terminated ethyleneoxide dimethylsiloxane, ethyleneoxide ABA block copolymer, dimethylsiloxane-(ethylene oxide) block copolymer, dimethylsiloxane-(25-30% ethylene oxide) block copolymer, and combinations thereof.

In an aspect, additive content my include about 0.1 to about −60% w/w, about 1 to about 50% w/w. about 5% to about 40% w/w. about 10% to about 30% w/w, or about 15% to about 25% w/w of the drug eluting material, e.g., 1, 2, 3, 4, 5, 6, 7, 8, 9, 10, 15, 20, or 25% w/w, or any range, integer or fraction of an integer between 1% and 25% w/w. For example, a silicone-based matrix may have additive content of from 0.5 to 25% w/w, 1 to 23% w/w. 2 to 20% w/w, 3 to 18% w/w. 5 to 15% w/w, 8 to 12% w/w, or 10 w/w %, or any combination of upper and lower bounds of any range of concentrations between 1 and 25% w/w.

In an aspect, the drug eluting material may include a specific drug loading content relative to the type of drug eluting polymer matrix used. In one aspect, the drug eluting material is a silicone-based matrix and has drug loading content of 1 wt. % to 60 wt. %, e.g., 1, 2, 3, 4, 5, 6, 7, 8, 9, 10, 15, 20, 25, 30, 35, 40, 45, 50, 55, or 60 wt. %, or any range, integer or fraction of an integer between 1 wt. % and 60 wt. %. For example, a silicone-based matrix may have a drug loading content of from 5 wt. % to 55 wt. %, 10 wt. % to 50 wt. %, 15 wt. % to 45 wt. %, 20 wt. % to 40 wt. %, 25 wt. % to 35 wt. %, 28 wt. % to 32 wt. %, or any combination of upper and lower bounds of any range of concentrations between 1 wt. % to 60 wt. %. In one aspect, the drug eluting material is an organic-based matrix and has drug loading content of 1 wt. % to 75 wt. %, e.g., 1, 2, 3, 4, 5, 6, 7, 8, 9, 10, 15, 20, 25, 30, 35, 40, 45, 50, 55, 60, 65, 70, 75 wt. %, or any range, integer or fraction of an integer between 1 wt. % and 60 wt. %. For example, an organic-based matrix may have a drug loading content of from 5 wt. % to 70 wt. %, 10 wt. % to 65 wt. %, 15 wt. % to 60 wt. %, 20 wt. % to 55 wt. %, 25 wt. % to 50 wt. %, 30 wt. % to 45 wt. %, 35 wt. % to 40 wt. %, or any combination of upper and lower bounds of any range of concentrations between 1 wt. % to 75 wt. %.

In an aspect, the drug eluting material may be selected from an organic hydrogel-based matrix containing polyethers, acrylics, silicones including medical grade silicones, or a derivative thereof, and combinations thereof. Non-limiting examples of silicones that may be used include liquid silicone rubber, silicone adhesive, silicone foam, silicone dispersion, and combinations thereof.

In an aspect, the drug eluting material may be modified by addition of catalytic additives that modify the cure rate of the material. In one aspect, the material is allowed to cure at about room temperature for about 12-36, about 18-30, about 20-28, about 22-26, or about 24 hours. In one aspect, the cure rate may be modified using catalytic additives.

In an aspect, the drug eluting material may be modified by applying a method of curing to achieve a tailored elution profile. Non-limiting examples of such curing methods include injection molding, curing using a heat gun, room temperature curing, curing at 30-40° C., e.g., 30, 31, 32, 33, 34, 35, 36, 37, 38, 39, or 40° C., or any range, integer or fraction of an integer between 30-40° C., or at a gradient of temperatures increasing or decreasing in a range of from 20-40° C., including any range, integer or fraction of an integer between 20-40° C.

In an aspect, the drug eluting material may be loaded with one or more of glucocorticoids, nonsteroidal anti-inflammatory drugs (NSAIDs), immunosuppressants, and antioxidants.

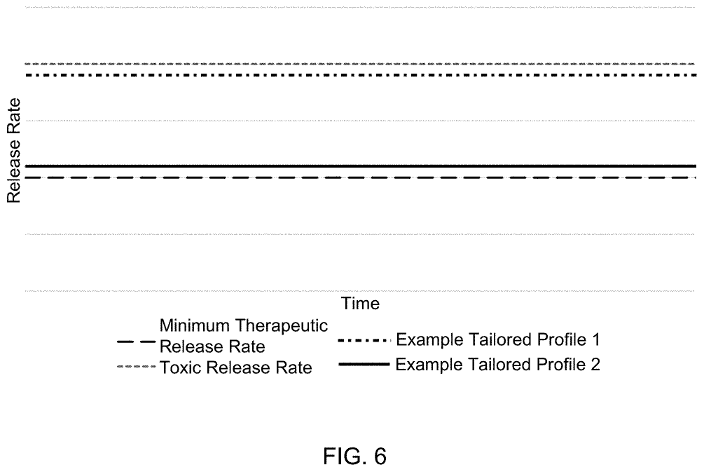

Any of the foregoing aspects may be used alone or in combination with each other to tailor the elution of one or more therapeutic agents. In one aspect, the drug eluting material has a steady state release profile. As used herein, the phrase “steady state release profile” refers to a release profile of a therapeutic agent at a steady release rate between its minimum therapeutic release rate and its toxic release rate for a period at least as long as the intended life of the sensor system. FIG. 6 illustrates two non-limiting examples of steady state release profiles according to the present disclosure.

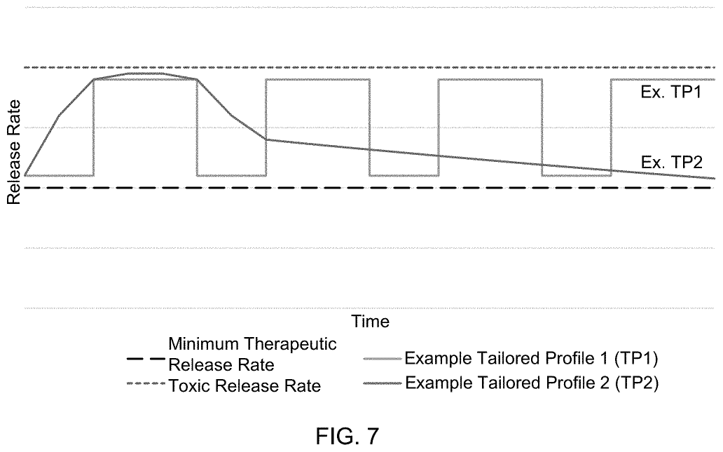

In a tailored elution profile according to the present disclosure, the release rate will depend on the therapeutic agent that is used. The release rate will be between a minimum therapeutic release rate of the therapeutic agent that is used and its toxic release rate. In a non-limiting and illustrative example, for a therapeutic agent having a minimum therapeutic release rate of 0.30 μg/day and toxic release rate of 30 μg/day, the tailored elution profile may include a release rate ranging from 0.30 μg/day to 30 μg/day, including any range, integer or fraction of an integer between 0.30 μg/day to 30 μg/day, e.g., 0.6 μg/day to 25 μg/day, 0.8 μg/day to 20 μg/day, 1 μg/day to 15 μg/day, 1.2 μg/day to 10 μg/day, 1.5 μg/day to 6 μg/day, or any combination of upper and lower bounds of any range of rates between 0.30 μg/day to 30 μg/day. FIG. 7 illustrates two non-limiting examples of tailored elution profiles according to the present disclosure.

In some aspects, the sensor 100 may include any of the foregoing aspects that tailor elution of drugs that interact with degradative species without compromising signal integrity or performance of the sensor. In some non-limiting aspects, the drug may be dexamethasone, triamcinolone, betamethasone, methylprednisolone, beclometasone, fludrocortisone, derivatives thereof, and analogs thereof, a glucocorticoid, or an anti-inflammatory drug (e.g., a non-steroidal anti-inflammatory drug including but not limited to acetylsalicylic acid, isobutylphenylpropanoic acid). Accordingly, the drug eluting material according to the present disclosure may advantageously extend the lifetime of implantable sensors.

In some non-limiting aspects, a sensor 100 for measurement of an analyte (e.g., glucose) in a medium (e.g., interstitial fluid) within a living animal (e.g., a human) may include a sensor housing 102 and an analyte indicator 106. In some aspects, the analyte indicator may include one or more indicator molecules 104, which may be distributed throughout the analyte indicator 106. In some aspects, the indicator molecules 104 may be configured to reversibly bind the analyte. In some aspects, the analyte indicator 106 may cover at least a portion of the sensor housing 102. In some aspects, the sensor 100 may include a light source 108 (e.g., within the sensor housing 102) configured to emit excitation light 329. In some aspects, the indicator molecules 104 may configured to be irradiated by the excitation light 329 and emit light 331 indicative of the amount of the analyte in the medium within the living animal. In some aspects, the sensor 100 may include a photodetector 224 (e.g., within the sensor housing 102) that is sensitive to light 331 emitted by the one or more indicator molecules 104 and configured to generate a signal indicative of the amount of the analyte in the medium within the living animal.

In some aspects, the at least one drug eluting material may include a membrane, mesh, nylon, fabric, polymer material, sponge, or other pore-containing material. In some aspects, one or more of the foregoing aspects may be incorporated into the analyte indicator 106 that may cover at least a portion of the sensor housing 102. In some aspects, the drug eluting polymer matrix may cover a portion of the sensor housing 102.

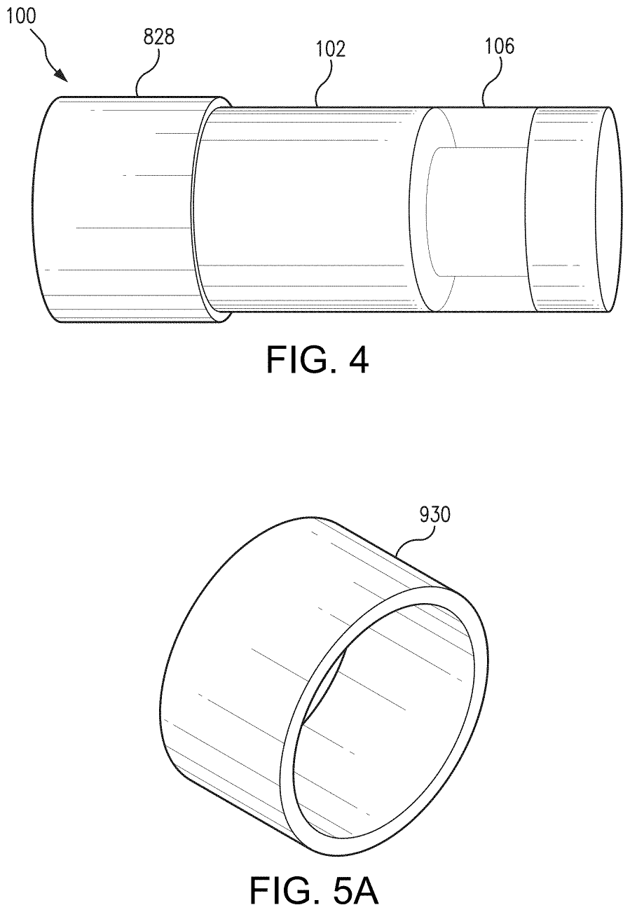

In some non-limiting aspects, the drug-eluting polymer matrix may be applied to the sensor housing 102 via dip coating. FIG. 4 illustrates a sensor 100 having a dip coated drug-eluting polymer matrix 828. In some aspects, as illustrated in FIG. 4, the dip coated drug-eluting polymer matrix 828 may cover a portion of the sensor housing 102. However, this is not required, and, in alternative aspects, the dip coated drug-eluting polymer matrix 828 may cover a different portion of the sensor housing 102 or the entire sensor housing 102. In some non-limiting aspects, as an alternative to dip coating, the drug-eluting polymer matrix may be applied to the sensor housing 102 via spray coating.

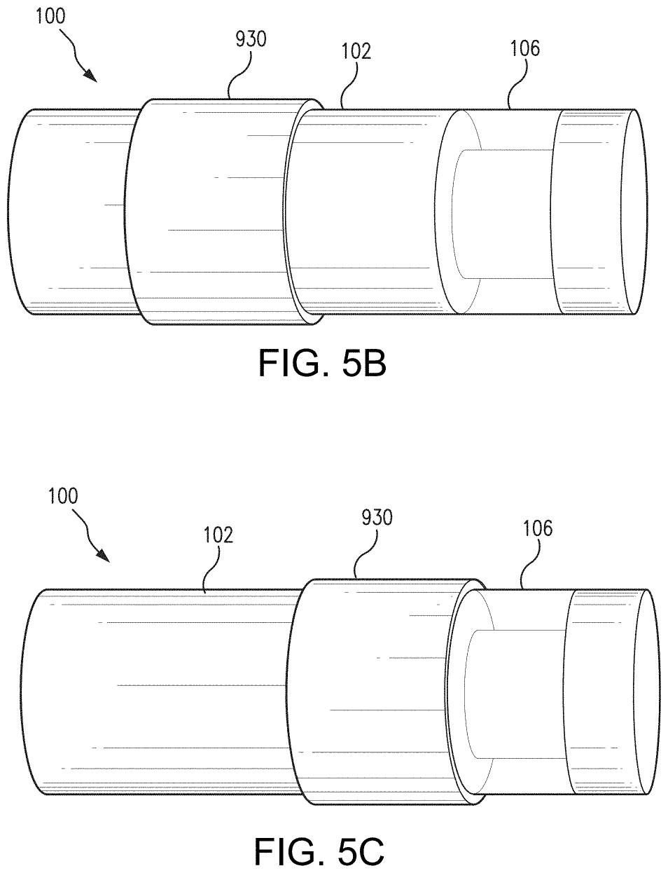

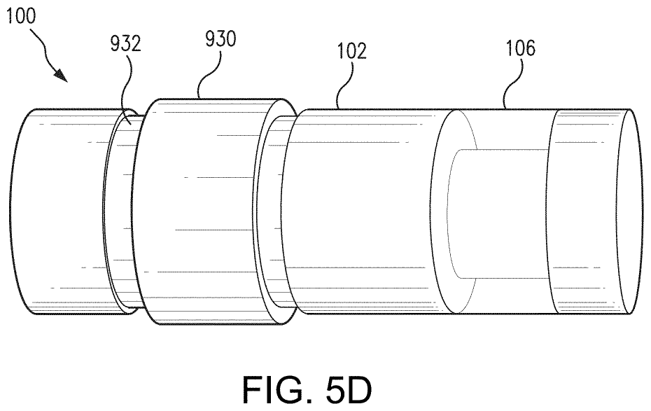

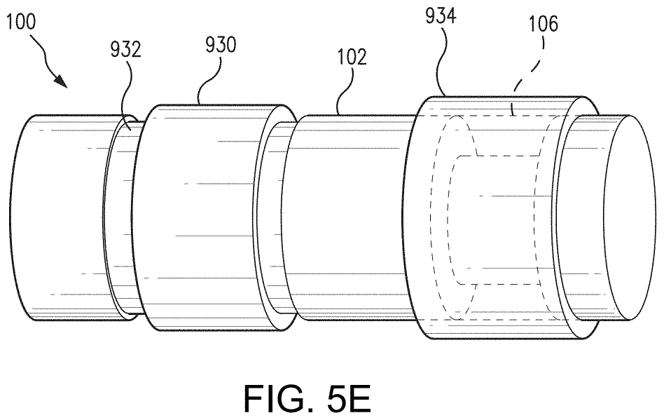

In some non-limiting aspects, as an alternative to dip coating, the drug-eluting polymer matrix may be applied to the sensor housing 102 via spray coating. In some non-limiting aspects, as an alternative to a dip or spray coated drug-eluting polymer matrix, the drug-eluting polymer matrix may have a pre-formed shape such as, for example, a ring or sleeve. Other pre-formed shapes are possible, such as, for example and without limitation, a shell (e.g., conformal shell), cylinder, or any suitable monolith (e.g. rectangular). FIG. 5A illustrates an example of a preformed, ring-shaped drug-eluting polymer matrix 930 that covers a portion of sensor housing 102. As illustrated in FIG. 5B, the ring-shaped drug-eluting polymer matrix 930 may wrap around a portion of the sensor housing 102. In some alternative aspects, the ring-shaped drug-eluting polymer matrix 930 may be wider or narrower than the ring-shaped drug-eluting polymer matrix 930 illustrated in FIG. 5B. For instance, in one non-limiting aspect, the preformed, ring-shaped drug-eluting polymer matrix 930 may have a width equal to the width of the sensor 100 (except for the portion constituting the polymer graft 106) and wrap around the entire width of the sensor 100. In another non-limiting aspect, as illustrated in FIG. 5C, the ring-shaped drug-eluting polymer matrix 930 may be located adjacent the polymer matrix 106. Although the ring-shaped drug-eluting polymer matrix 930 is located on one side of the polymer matrix 106 in aspect illustrated in FIG. 5C, the ring-shaped drug-eluting polymer matrix 930 could be located to the other side of the polymer matrix 106 or on both sides of the polymer matrix 930. In some non-limiting aspects, as illustrated in FIG. 5D, the sensor housing 102 may include a groove 932, and the ring-shaped drug-eluting polymer matrix 930 may be positioned in the groove 932. The edges of the groove 932 may assist in holding the ring-shaped drug-eluting polymer matrix 930 in place on the sensor housing 102.

In some non-limiting aspects, as illustrated in FIG. 5E, the sensor 100 may include a membrane 934 covering at least a portion of the analyte indicator. In one non-limiting aspect, the membrane 934 may be an analyte permeable membrane. The membrane 934 may be positioned over the polymer graft 106 (and over any thin layer on the outside of the graft 106). The membrane 934 may be opaque and, therefore, perform a light-blocking function. In other words, the opaque nature of the membrane 934 may serve the function of effectively blocking the extraneous light from over stimulating the indicator molecules 104 of the graft 106. In some non-limiting aspects, the opaque membrane 934 may be physically attached over the graft 106 after boring an additional, smaller well into the capsule/housing 102.

In some aspects, the membrane 934 may be porous. In other words, the membrane 934 may be structured so that it channels one or more analytes (e.g., glucose) to the graft 106. For example, in one non-limiting aspect, the membrane 934 may have small pores (e.g., pores having a pore size of microns or less) that block white blood cells (e.g., neutrophils), which are between 6 and 12 microns in diameter, from reaching the underlying graft 106 to attack it. The small pores, however, would at the same time be large enough to allow the analyte to reach the graft 106. In this way, a porous membrane 934 having small pores would increase sensor longevity while not affecting the ability of the sensor 100 to measure the analyte.

In some aspects, the opaque membrane 934 may be made from a material that does not react adversely to the body's defenses. In non-limiting aspects, the material from which the opaque membrane 934 is made may additionally be both porous (e.g., to allow and analyte, such as glucose, to flow through it) and opaque (e.g., to prevent light from traveling through it). For example, in some aspects, the membrane (e.g., mesh) material may be a material such as nylon, cellulose acetate, polypropylene (PP), polyvinyl alcohol (PVA), polybutylene terephthalate (PBT), polyether ether ketone (PEEK), polyanhydride, polyamide, polyvinylchloride (PVC), polyethersulfone (PES), polyethylene terephthalate (PET), polyvinylidene difluoride (PVDF), polytetrafluoroethylene (PTFE), and/or polycarbonate.

In some aspects, the membrane 934 may be a porous, opaque diffusion membrane that is configured to: substantially prevent white blood cells from passing through the membrane, permit an analyte of interest to pass through the membrane to the graft, and substantially prevent transmission of light of at least a specified wavelength or range of wavelengths through the membrane.

In some aspects, to enhance biocompatibility and/or hydrophilicity, the membrane 934 may comprise an additional thin layer, and/or the membrane 934 may comprise multiple mesh layers. In some aspects, the membrane 934 and the one or more therapeutic agents may have an additive effect in reducing oxidation of the analyte indicator.

One or more types of therapeutic agents may be dispersed within the drug eluting material (e.g., a polymer matrix). In some aspects, the one or more the drugs eluted from the drug eluting material having tailored elution properties may reduce or stop the migration of neutrophils from entering the insertion site and, thus, reduce or stop the production of hydrogen peroxide and fibrotic encapsulation. In some aspects, the one or more drugs may be provided in the analyte indicator 106 (e.g., polymer graft). In some aspects, the one or more drugs may interact and/or react with degradative species. In some aspects, the one or more drugs may neutralize the degradative species. In some aspects, the one or more drugs may bind to the degradative species. In some aspects, the one or more drugs may sequester the degradative species so as to inhibit, reduce, and/or prevent degradation of the analyte indicator 106 by the degradative species. Accordingly, in some aspects, the one or more drugs reduce deterioration of the analyte indicator 106.

A sensor having one or more drugs configured to be eluted from a drug eluting material having tailored elution properties may have improved performance over a sensor that does not include a drug eluting material having tailored elution properties. For instance, in some non-limiting aspects, the drug eluting material having tailored elution properties may improve the longevity and functionality of the sensor 100.

### Examples

Exemplary and non-limiting formulations according to the present disclosure were manufactured as follows:

| Group | Additives |
|---|---|
| A1 | Hydroxypropyl methylcellulose |
| A2 | Acryloxy Terminated Ethyleneoxide Dimethysiloxane, |
| | Ethyleneoxide ABA Block Copolymer |
| A3 | Dimethysiloxane-(25-30% Ethylene Oxide) Block Copolymer |

| Group | Silicone Grade | Type |
|---|---|---|
| SG1 | Nusil Med-4850 | Liquid Silicone Rubber |
| SG2 | Nusil Med3-4213 | Silicone Adhesive |
| SG3 | Nusil Med-4830 | Liquid Silicone Rubber |
| SG4 | Nusil Med-2310 | Silicone Foam |
| SG5 | Nusil Med-6670 | Silicone Dispersion |

| | | Drug | | Additive | |
|---|---|---|---|---|---|
| Sample | Silicone | Content | | Content | |
| Group | Grade | (w/w) | Additives | (w/w) | Cure Method |
| 1 | SG1 | 32% | None | N/A | Injection |
| | | | | | Molded |
| 2 | SG1 | 32% | A1 | 5% | Injection |
| | | | | | Molded |
| 3 | SG1 | 32% | A1 | 10% | Injection |
| | | | | | Molded |
| 4 | SG1 | 48% | None | N/A | Injection |
| | | | | | Molded |
| 5 | SG1 | 30% | None | N/A | Room |
| | | | | | Temperature |
| 6 | SG1 | 30% | None | N/A | 32 C. Cure |
| 7 | SG1 | 30% | None | N/A | 37 C. Cure |
| 8 | SG1 | 30% | None | N/A | Heat Gun |
| 9 | SG1 | 38% | None | N/A | Room |
| | | | | | Temperature |
| 10 | SG1 | 43% | None | N/A | Heat Gun |
| 11 | SG2 | 10% | A2 | 5% | Room |
| | | | | | Temperature |
| 12 | SG2 | 20% | A2 | 5% | Room |
| | | | | | Temperature |
| 13 | SG2 | 30% | A2 | 5% | Room |
| | | | | | Temperature |
| 14 | SG2 | 10% | A2 | 2% | Room |
| | | | | | Temperature |
| 15 | SG2 | 20% | A2 | 2% | Room |
| | | | | | Temperature |
| 16 | SG2 | 30% | A2 | 2% | Room |
| | | | | | Temperature |
| 17 | SG2 | 10% | A2 | 1% | Room |
| | | | | | Temperature |
| 18 | SG2 | 20% | A2 | 1% | Room |
| | | | | | Temperature |
| 19 | SG2 | 30% | A2 | 1% | Room |
| | | | | | Temperature |
| 20 | SG2 | 10% | A3 | 1% | Room |
| | | | | | Temperature |
| 21 | SG2 | 20% | A3 | 1% | Room |
| | | | | | Temperature |
| 22 | SG2 | 30% | A3 | 1% | Room |
| | | | | | Temperature |
| 23 | SG2 | 10% | A3 | 2% | Room |
| | | | | | Temperature |
| 24 | SG2 | 20% | A3 | 2% | Room |
| | | | | | Temperature |
| 25 | SG2 | 30% | A3 | 2% | Room |
| | | | | | Temperature |
| 26 | SG2 | 10% | A3 | 5% | Room |
| | | | | | Temperature |
| 27 | SG2 | 20% | A3 | 5% | Room |
| | | | | | Temperature |
| 28 | SG2 | 30% | A3 | 5% | Room |
| | | | | | Temperature |
| 29 | SG2 | 48% | None | N/A | Room |
| | | | | | Temperature |
| 30 | SG2 | 48% | None | N/A | Heat Gun |
| 31 | SG2 | 30% | None | N/A | Room |
| | | | | | Temperature |
| 32 | SG2 | 30% | None | N/A | Heat Gun |
| 33 | | 30% | None | N/A | Room |
| | | | | | Temperature |
| 34 | SG4 | 30% | None | N/A | Room |
| | | | | | Temperature |
| 35 | SG5 | 30% | None | N/A | Room |
| | | | | | Temperature |
| 36 | SG5 | 50% | None | N/A | Room |
| | | | | | Temperature |

Drug delivery rates based on various test methods have been generated. The test most similar to in-vivo drug delivery kinetics is performed in a PBS based solution. Illustrative sample groups were tested in PBS and the release rates are presented below. The presents demonstrate that release rates can be tailored relative to the control samples, e.g., by a factor of 2-fold, 3-fold, 4-fold, 5-fold or more. Accordingly, the release profile for a given therapeutic agent can be tailored to remain between the minimum therapeutic release rate and the maximum toxic level of the therapeutic agent for a predetermined time period, e.g., the life of the sensor.

| Sample Group | Release Rate (μg/day) |
|---|---|
| 1 (control samples) | 0.50 |
| 29 | 0.43 |
| 31 | 0.90 |
| 33 | 1.20 |
| 4 | 2.49 |
| 5 | 1.86 |

Aspects of the present invention have been fully described above with reference to the drawing figures. Although the invention has been described based upon these preferred aspects, it would be apparent to those of skill in the art that certain modifications, variations, and alternative constructions could be made to the described aspects within the spirit and scope of the invention. For example, although in some aspects, the analyte sensor 100 may be an optical sensor, this is not required, and, in one or more alternative aspects, the analyte sensor may be a different type of analyte sensor, such as, for example, an electrochemical sensor, a diffusion sensor, or a pressure sensor. Also, although in some aspects, the analyte sensor 100 may be an implantable sensor, this is not required, and, in some alternative aspects, the analyte sensor may be a transcutaneous sensor having a wired connection to an external transceiver. For example, in some alternative aspects, the analyte sensor 100 may be located in or on a transcutaneous needle (e.g., at the tip thereof). In these aspects, instead of wirelessly communication using an antenna (e.g., inductive element 114), the analyte sensor may communicate with the external transceiver using one or more wires connected between the external transceiver and a transceiver transcutaneous needle including the analyte sensor. For another example, in some alternative aspects, the analyte sensor may be located in a catheter (e.g., for intravenous blood glucose monitoring) and may communicate (wirelessly or using wires) with an external transceiver.

What is claimed is:

1. A sensor for measurement of an analyte in a medium within a living animal, the sensor comprising:
a sensor housing;
an analyte indicator covering at least a portion of the sensor housing; and
a drug eluting material having tailored elution properties comprising a drug that reduces deterioration of the analyte indicator, wherein the drug eluting material is incorporated in and/or in close proximity to the analyte indicator, and the drug eluting material is configured to release the drug according to a tailored elution profile.

2. The sensor of claim 1, wherein the sensor is implantable within a living animal.

3. The sensor of claim 1, wherein the drug eluting material comprises at least one drug eluting polymer matrix covering at least a portion of the sensor housing, and the drug is dispersed within the drug eluting polymer matrix.

4. The sensor of claim 1, wherein the tailored elution profile includes a release rate that is between a minimum therapeutic release rate and a toxic release rate of the drug.

5. The sensor of claim 1, wherein the drug eluting material comprises 0.1-60% w/w of an additive.

6. The sensor of claim 1, wherein the drug eluting material comprises an additive selected from: a hydroxypropyl methylcellulose; a polyalkylene glycol; a polyalkylene oxide; a polyether or a copolymer of polyethers; di-block, tri-block, grafted, post-functionalized polyether-siloxane copolymers; copolymers thereof, and combinations thereof.

7. The sensor of claim 1, wherein the drug eluting material comprises an additive selected from: a hydroxypropyl methylcellulose; polyethylene glycol; polypropylene glycol; polyethylene oxide; polypropylene oxide; copolymers thereof, and combinations thereof.

8. The sensor of claim 1, wherein the drug eluting material comprises a silicone-based matrix and 1 wt. % to 60 wt. % of said drug.

9. The sensor of claim 1, wherein the drug eluting material comprises an organic-based matrix and 1 wt. % to 75 wt. % of said drug.

10. The sensor of claim 1, wherein the drug eluting material is selected from an organic hydrogel-based matrix containing polyethers, acrylics, silicones including medical grade silicones, derivatives thereof, and combinations thereof.

11. The sensor of claim 1, wherein the drug eluting material comprises liquid silicone rubber, silicone adhesive, silicone foam, silicone dispersion, and combinations thereof.

12. The sensor of claim 1, wherein the drug eluting material comprises one or more catalytic additives that modify the cure rate of the drug eluting material.

13. The sensor of claim 1, wherein the drug eluting material is injection molded, cured using a heat gun, cured at room temperature, or cured at 30-40° C.

14. The sensor of claim 1, wherein the drug eluting material comprises one or more of glucocorticoids, nonsteroidal anti-inflammatory drugs (NSAIDs), immunosuppressants, and antioxidants.

15. The sensor of claim 1, wherein the drug eluting material is configured to elute the drug to interact or react with a degradative species without compromising signal integrity or performance of the sensor device, and the degradative species is hydrogen peroxide, a reactive oxygen species, a reactive nitrogen species, or a free radical.

16. The sensor of claim 1, wherein the drug eluting material has a preformed shape.

17. The sensor of claim 16, wherein the preformed shape is a ring, a sleeve, a conformal shell, a cylinder, or a monolith.

18. The sensor of claim 1, wherein the drug eluting material comprises acetylsalicylic acid.

19. The sensor of claim 1, wherein the drug eluting material comprises isobutylphenylpropanoic acid.

20. The sensor of claim 1, wherein the drug is dexamethasone, triamcinolone, betamethasone, methylprednisolone, beclometasone, fludrocortisone, a derivative thereof, an analog thereof, or a combination of two or more thereof.

21. The sensor of claim 1, wherein the analyte indicator is a graft including indicator molecules.

22. The sensor of claim 1, further comprising a layer of a catalyst capable of converting hydrogen peroxide into water and oxygen on at least a portion of the analyte indicator.

23. The sensor of claim 1, further comprising a membrane covering at least a portion of the analyte indicator.

24. The sensor of claim 23, wherein the membrane is a porous, opaque diffusion membrane.

25. A method of fabricating a sensor for measurement of an analyte in a medium within a living animal, the method comprising:
applying an analyte indicator to a sensor housing of the sensor such that the applied analyte indicator covers at least a portion of the sensor housing, and
applying a drug eluting material having tailored elution properties comprising a drug that reduces deterioration of the analyte indicator such that the applied drug eluting material is incorporated to the sensor in and/or in close proximity to the analyte indicator, wherein the drug eluting material is configured to release the drug according to a tailored elution profile and reduce deterioration of the analyte indicator.
